(19) United States
(12) Patent Application Publication
(10) Pub. No.: US 2019/0112256 A1
(43) Pub. Date: Apr. 18, 2019

(54) POLYETHERAMINES BASED ON 1,3-DIALCOHOLS

(71) Applicant: BASF SE, Ludwigshafen am Rhein (DE)

(72) Inventors: Bjoern Ludolph, Ludwigshafen (DE); Sophia Ebert, Ludwigshafen (DE); Christian Eidamshaus, Ludwigshafen (DE); Stefano Scialla, Cincinnati, OH (US); Brian Joseph Loughnane, Cincinnati, OH (US); Karie Marie Henke, Cincinnati, OH (US); Gayle Marie Frankenbach, Cincinnati, OH (US); Jay Frank Nash, Cincinnati, OH (US); Michael Patrick Hayes, Cincinnati, OH (US); Manuel G. Venegas, Cincinnati, OH (US)

(21) Appl. No.: 16/087,732

(22) PCT Filed: Mar. 14, 2017
(86) PCT No.: PCT/EP2017/055943

§ 371 Date: Sep. 24, 2018

(30) Foreign Application Priority Data
Mar. 24, 2016 (EP) 16162177.6

(51) Int. Cl. C07C 217/08; C07C 217/12; C08G 65/333

(52) U.S. Cl. CPC C07C 217/08; C07C 213/02; C08G 65/333; C07C 217/12

(57) ABSTRACT

Described herein is an etheramine mixture including at least 90% by weight, based on the total weight of the etheramine mixture, of an amine of Formula (I) and/or (II),

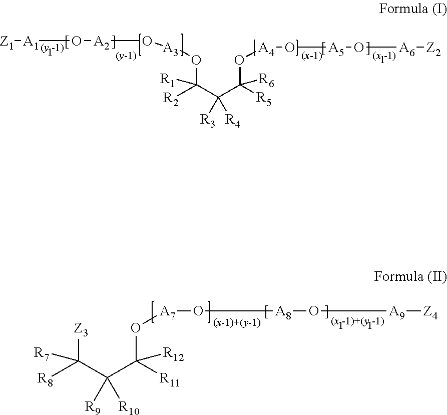

wherein R₁-R₁₂ are independently selected from H, alkyl, cycloalkyl, aryl, alkylaryl, or arylalkyl, wherein at least one of R₁-R₆ and at least one of R₇-R₁₂ is different from H, wherein A₁-A₉ are independently selected from linear or branched alkanediyl groups having 2 to 18 carbon atoms,
wherein Z₁-Z₄ are independently selected from —OH and linear —OCH₂CH₂CH₂NH₂, wherein the degree of amination of each of the etheramines of Formula (I) and Formula (II) is at least 50%, and wherein the sum of x+y is in the range of from 2 to 200, wherein x≥1 and y≥1; and x₁+y₁ is in the range of from 2 to 200, wherein x₁≥1 and y₁≥1.

This invention relates to polyetheramines with linear oxypropylamine groups based on 1,3-dialcohols.

Due to the increasing popularity of easy-care fabrics made of synthetic fibers as well as the ever increasing energy costs and growing ecological concerns of detergent users, the once popular hot water wash has now taken a back seat to washing fabrics in cold water. Many commercially available laundry detergents are even advertised as being suitable for washing fabrics at 40° C. or 30° C. or even at room temperature. To achieve satisfactory washing result at such low temperatures, results comparable to those obtained with hot water washes, the demands on lowtemperature detergents are especially high.

It is known to include certain additives in detergent compositions to enhance the detergent power of conventional surfactants so as to improve the removal of grease stains at temperatures of 60° C. and below.

WO 86/07603 discloses that detergent composition comprising an aliphatic amine compound, in addition to at least one synthetic anionic and/or nonionic surfactant, are known and have led to improved cleaning results even at low wash temperatures.

Also, the use of linear, alkyl-modified (secondary) alkoxypropylamines in laundry detergents to improve cleaning at low temperatures is known (WO90/03423). These known laundry detergents, however, are unable to achieve satisfactory cleaning when laundry is washed at cold temperatures.

Furthermore, the use of linear, primary polyoxyalkyleneamines (e.g., Jeffamine® D-230) to stabilize fragrances in laundry detergents and provide longer lasting scent is also known (WO2009/065738). Also, the use of high-molecular-weight (molecular weight of at least about 1000), branched, trifunctional, primary amines (e.g., Jeffamine@ T-5000 polyetheramine) to suppress suds in liquid detergents is known (WO01/76729).

Additionally, WO 2011/087793 reads on etheramine mixtures comprising at least 10 wt % of an alkoxylated monoether amine based on polyhydric alcohols containing 2 to 4 hydroxyl groups as the starting compound. A process for the manufacture of these etheramine mixtures is also disclosed. These products find an application as a curing agent or as a raw material in the synthesis of polymers.

Furthermore, WO 2014/154783 discloses polyetheramines, wherein at least half of the terminal groups are amine groups, based on 1,3-dialcohols and their use in cleaning compositions.

There is a continuous need for cleaning compositions that remove grease stains from fabrics and other soiled materials, as grease stains are challenging stains to remove. Conventional cleaning compositions directed to grease removal frequently utilize various amine compounds which tend to show strong negative impacts on whiteness. As a consequence there is still a continual need for improved amine compositions which provide improved grease removal from fabrics and other soiled materials and at the same time do not negatively impact the clay cleaning.

It was an object of the present invention to provide compounds which would improve the washing performance of detergents at low temperatures, i.e. at temperatures as low as 30° C. or even lower.

This goal was achieved by the present invention as described herein below and as reflected in the claims.

The present invention relates to an etheramine mixture comprising at least 90% by weight, based on the total weight of the etheramine mixture, of an amine of Formula (I) and/or (II),

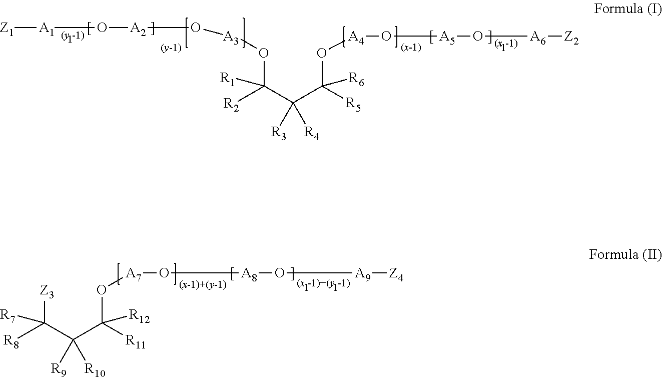

wherein R₁-R₁₂ are independently selected from H, alkyl, cycloalkyl, aryl, alkylaryl, or arylalkyl, wherein at least one of R₁-R₆ and at least one of R₇-R₁₂ is different from H, wherein A₁-A₉ are independently selected from linear or branched alkanediyl groups having 2 to 18 carbon atoms, preferably 2-10 carbon atoms, most preferably 2-5 carbon atoms, wherein Z₁-Z₄ are independently selected from —OH or linear —OCH₂CH₂CH₂NH₂, wherein the degree of amination of each of the etheramines of Formula (I) and Formula (II) is equal to or greater than 50%, and wherein the sum of x+y is in the range of about 2 to about 200, wherein x≥1 and y≥1; and x₁+y₁ is in the range of about 2 to about 200, preferably 2-20, most preferably 2-10, wherein x₁≥1 and y₁≥1.

The etheramine mixture according to the present invention of Formula (I) and (II) having equal to or more than half of the Z₁-Z₄ groups comprise linear 1-oxy-3-propylamine groups (OCH₂CH₂CH₂NH₂) provides improved washing performance of detergents.

In one embodiment of the present invention, the sum of x and y may be in the range of 2 to 20, 2 to 10, 3 to 8 or 4 to 6.

In one embodiment of the present invention, the sum of x₁ and y₁ may be in the range of 2 to 20, 2 to 10, 3 to 8 or 2 to 4.

In one embodiment of the present invention, the etheramine mixture may comprise at least 95% by weight, based on the total weight of the etheramine mixture, of the amine of Formula (I) and/or (II).

In another embodiment of the present invention, A₁-A₉ may independently be selected from the group consisting of ethylene, propylene, and butylene. For example, each of A₁-A₉ is propylene.

In Formula (I) or (II), R₁, R₂, R₅, R₆, R₇, R₈, R₁₁, and R₁₂ may be H, and R₃, R₄, R₉, and R₁₀ may independently be selected from C1-16 alkyl and aryl.

In one embodiment of the present invention, in Formula (I) or (II), R₁, R₂, R₅, R₆, R₇, R₈, R₁₁, and R₁₂ may be H, and R₃, R₄, R₉, and R₁₀ may independently be selected from a butyl group, an ethyl group, a methyl group, a propyl group, and a phenyl group.

In one specific embodiment of the present invention, in Formula (I) or (II), R3 and R9 may be each an ethyl group, R₁, R₂, R₅, R₆, R₇, R₈, R₁₁, and R₁₂ may be each H, and/or R4 and R10 may be each a butyl group.

The polyetheramine of Formula (I) or Formula (II) may have a weight average molecular weight of about 290 to about 1000 grams/mol, 300 to about 700 grams/mol, or 300 to about 450 grams/mol.

The etheramine mixture comprising at least 90% by weight, based on the total weight of the etheramine mixture, of an etheramin of Formula (I) and/or (II) having equal to or more than half of the Z₁-Z₄ groups comprise linear 1-oxy-3-propylamine groups (—OCH₂CH₂CH₂NH₂) may be obtainable by the following process comprising the steps:
a) the reaction of 1,3-diols of Formula (III) with C₂-C₁₈ alkylene oxides, wherein the molar ratio of 1,3-diol to C₂-C₁₈ alkylene oxides is in the range of 1:2 to 1:10,

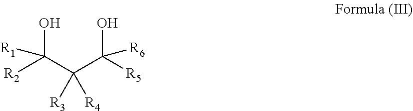

with R₁-R₆ are independently of one another H, alkyl, cycloalkyl, aryl, alkylaryl, arylalkyl and at least one group selected from R₁-R₆ is different from H, followed by
b) reductive cyanoethylation of the alkoxylated 1,3-diols.

Generally, as used herein, the term “obtainable by” means that corresponding products do not necessarily have to be produced (i.e. obtained) by the corresponding method or process described in the respective specific context, but also products are comprised which exhibit all features of a product produced (obtained) by said corresponding method or process, wherein said products were actually not produced (obtained) by such method or process. However, the term “obtainable by” also comprises the more limiting term “obtained by”, i.e. products which were actually produced (obtained) by a method or process described in the respective specific context.

In one embodiment of the present invention, this etheramine mixture comprising at least 95% by weight, based on the total weight of the etheramine mixture, of the amine of Formula (I) and/or Formula (II).

In one embodiment of the present invention, the molar ratio of 1,3-diol to C₂-C₁₈ alkylene oxides may be in the range of 1:1 to 1:8, or 1:2 to 1:7, or 1:3 to 1:6, or 1:4 to 1:5 In one embodiment of the present invention, the C₂-C₁₈ alkylene oxides may be selected from the group consisting of ethylene oxide, propylene oxide, butylene oxide and a mixture thereof, for example C₂-C₁₈ alkylene oxide is propylene oxide.

In one embodiment of the present invention, in the 1,3-diol of Formula (III) R₁, R₂, R₅, R₆ are H and R₃, R₄ may be C1-16 alkyl or aryl.

The 1,3-diol of Formula (III) may be selected from the group consisting of 2-butyl-2-ethyl-1,3-propanediol, 2-methyl-2-propyl-1,3-propanediol, 2-methyl-2-phenyl-1,3-propanediol, 2,2-dimethyl-1,3-propanediol, and 2-ethyl-1,3-hexanediol.

Step a): alkoxylation

Substituted 1,3 diols (Formula III) may be synthesized, e.g., according WO10026030, WO10026066, WO09138387, WO09153193, or WO10010075.

Suitable 1,3-diols (Formula III) comprise for example: 2,2-dimethyl-1,3-propane diol, 2-butyl-2-ethyl-1,3-propane diol, 2-pentyl-2-propyl-1,3-propane diol, 2-(2-methyl)butyl-2-propyl-1,3-propane diol, 2,2,4-trimethyl-1,3-propane diol, 2,2-diethyl-1,3-propane diol, 2-methyl-2-propyl-1,3-propane diol, 2-ethyl-1,3-hexane diol, 2-phenyl-2-methyl-1,3-propane diol, 2-methyl-1,3-propane diol, 2-ethyl-2-methyl-1,3 propane diol, 2,2-dibutyl-1,3-propane diol, 2,2-di(2-methylpropyl)-1,3-propane diol, 2-isopropyl-2-methyl-1,3-propane diol, etc.

Preferred 1,3-diols comprise 2-butyl-2-ethyl-1,3-propanediol, 2-methyl-2-propyl-1,3-propanediol, 2-methyl-2-phenyl-1,3-propanediol.

Alkoxylated 1,3-diols may be obtained by reaction of 1,3-diols (Formula III) with alkylene oxides.

The alkoxylated 1,3-diols may be prepared in a known manner by reaction of 1,3-diols with alkylene oxides. Suitable alkylene oxides comprise C₂-C₁₈ alkylene oxides like ethylene oxide, propylene oxide, butylene oxide, pentene oxide, hexene oxide, decene oxide, dodecene oxide etc.

Preferred C₂-C₁₈ alkylene oxides comprise ethylene oxide, propylene oxide, butylene oxide or a mixture thereof.

The 1,3-diols may be reacted with one single alkylene oxide or combinations of two or more different alkylene oxides. Using two or more different alkylene oxides, the resulting polymer can be obtained as a block-wise structure or a random structure.

The molar ratio of molar ratio of 1,3-diol to C₂-C₁₈ alkylene oxides at which the alkoxylation reaction is carried out may lie in the range of 1:2 to 1:10, preferably in the range of 1:3 to 1:8, even more preferably in the range of 1:4 to 1:6.

This reaction may be undertaken generally in the presence of a catalyst in an aqueous solution at a reaction temperature from about 70 to about 200° C. and preferably from about 80 to about 160° C. This reaction may be affected at a pressure of up to about 10 bar, and in particular up to about 8 bar.

Examples of suitable catalysts comprise basic catalysts such as alkali metal and alkaline earth metal hydroxides such as sodium hydroxide, potassium hydroxide and calcium hydroxide, alkali metal alkoxides, in particular sodium and potassium C₁-C₄-alkoxides, such as sodium methoxide, sodium ethoxide and potassium tert-butoxide, alkali metal and alkaline earth metal hydrides such as sodium hydride and calcium hydride, and alkali metal carbonates such as sodium carbonate and potassium carbonate. Preference is given to alkali metal hydroxides, particular preference being given to potassium hydroxide and sodium hydroxide. Typical use amounts for the base are from 0.05 to 10% by weight, in particular from 0.1 to 2% by weight, based on the total amount of diol and alkylene oxide.

Alkoxylation with x+y+x₁+y₁-2 C₂-C₁₈ alkylene oxides leads to structures as drawn in Formula IV and/or Formula V:

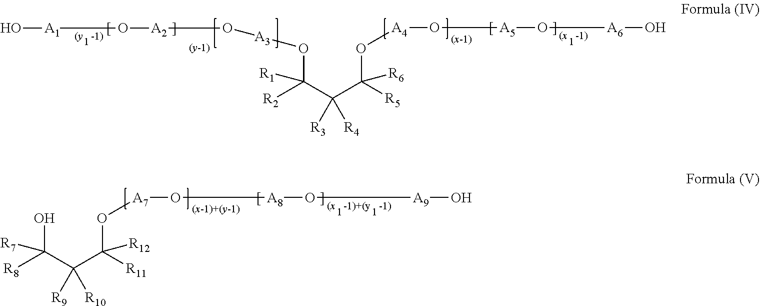

wherein R₁-R₁₂ are independently selected from H, alkyl, cycloalkyl, aryl, alkylaryl, or arylalkyl, wherein at least one of R₁-R₆ and at least one of R₇-R₁₂ is different from H,
wherein A₁-A₉ are independently selected from linear or branched alkanediyl groups having 2 to 18 carbon atoms, preferably 2-10 carbon atoms, most preferably 2-5 carbon atoms,
and wherein the sum of x+y is in the range of about 2 to about 200, wherein x≥1 and y≥1; and x₁+y₁ is in the range of about 2 to about 200, preferably 2-20, most preferably 2-10, wherein x₁≥1 and y₁≥1.

### Step b): Amination

Amination of the alkoxylated 1,3-diols may be carried out by reductive cyanoethylation, and leads to new structures with Formula I and/or (II):

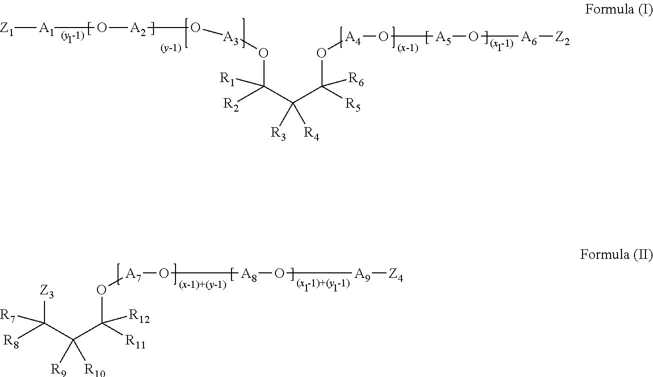

wherein R₁-R₁₂ are independently selected from H, alkyl, cycloalkyl, aryl, alkylaryl, or arylalkyl, wherein at least one of R₁-R₆ and at least one of R₇-R₁₂ is different from H, wherein A₁-A₉ are independently selected from linear or branched alkanediyl groups having 2 to 18 carbon atoms, preferably 2-10 carbon atoms, most preferably 2-5 carbon atoms, wherein Z₁Z₄ are independently selected from —OH or linear —OCH₂CH₂CH₂NH₂, wherein the degree of amination of each of the etheramines of Formula (I) and Formula (II) is equal to or greater than 50%, and wherein the sum of x+y is in the range of about 2 to about 200, wherein x≥1 and y≥1; and x₁+y₁ is in the range of about 2 to about 200, preferably 2-20, most preferably 2-10, wherein x₁≥1 and y₁≥1.

Polyetheramines according to Formula (I) and/or (II) are inter alia obtained by reductive cyanoethylation of the alkoxylated 1,3-diol mixture (Formula IV and V). The reductive cyanoethylation may be carried out by reaction of the alkoxylated 1,3-diol mixture (Formula IV and V) with acrylonitrile in the presence of a base followed by hydrogenation with hydrogen and a catalyst. The use of acrylonitrile leads to linear oxypropylamine end groups according to the present invention.

Suitable bases typically comprise alkaline hydroxides, and substituted ammonium hydroxide. Preferably, tetrakis(2-hydroxyethyl)ammonium hydroxide is used as a base.

As catalysts for hydrogenation the nitrile function to the corresponding amine, it is possible to use, in particular, catalysts which comprise one or more elements of the 8^{th} transition group of the Periodic Table (Fe, Co, Ni, Ru, Rh, Pd, Os, Ir, Pt), preferably Fe, Co, Ni, Ru or Rh, particularly preferably Co or Ni, in particular Co, as active component. A further preferred active component is Cu.

The abovementioned catalysts can be doped in the usual way with promoters, for example chromium, iron, cobalt, manganese, molybdenum, titanium, tin, metals of the alkali metal group, metals of the alkaline earth metal group and/or phosphorus.

As catalysts, preference can be given to using skeletal catalysts (also referred to as Raney® type, hereinafter also: Raney catalyst) which are obtained by leaching (activating) an alloy of hydrogenation-active metal and a further component (preferably Al). Preference is given to using Raney nickel catalysts or Raney cobalt catalysts.

Furthermore, supported Pd or Pt catalysts are preferably used as catalysts. Preferred support materials are activated carbon, Al20 3, Ti0₂, Zr0₂ and Si0₂. In a very preferred embodiment,

catalysts produced by reduction of catalyst precursors are used in the process of the invention.

The catalyst precursor comprises an active composition which comprises one or more catalytically active components, optionally promoters and optionally a support material.

The catalytically active components comprise oxygen-comprising compounds of the abovementioned metals, for example the metal oxides or hydroxides thereof, e.g. CoO, NiO, CuO and/or mixed oxides thereof.

For the purposes of the present patent application, the term “catalytically active components” is used for abovementioned oxygen-comprising metal compounds but is not intended to apply that these oxygen-comprising compounds are themselves catalytically active. The catalytically active components generally display catalytic activity in the reaction according to the invention only after reduction.

Particular preference is given to catalyst precursors such as the oxide mixtures which are disclosed in EP-A-0636409 and before reduction with hydrogen comprise from 55 to 98% by weight of Co, calculated as CoO, from 0.2 to 15% by weight of phosphorus, calculated as H₃PO₄, from 0.2 to 15% by weight of manganese, calculated as MnO₂, and from 0.2 to 5.0% by weight of alkali metal, calculated as M₂O (M=alkali metal), or oxide mixtures which are disclosed in EP-A-0742045 and before reduction with hydrogen comprise from 55 to 98% by weight of Co, calculated as CoO, from 0.2 to 15% by weight of phosphorus, calculated as H₃PO₄, from 0.2 to 15% by weight of manganese, calculated as MnO₂, and from 0.05 to 5% by weight of alkali metal, calculated as M₂O (M=alkali metal), or oxide mixtures which are disclosed in EP-A-696572 and before reduction with hydrogen comprise from 20 to 85% by weight of ZrO₂, from 1 to 30% by weight of oxygen-comprising compounds of copper, calculated as CuO, from 30 to 70% by weight of oxygen-comprising compounds of nickel, calculated as NiO, from 0.1 to 5% by weight of oxygen-comprising compounds of molybdenum, calculated as MoO₃, and from 0 to 10% by weight of oxygen-comprising compounds of aluminum and/or manganese, calculated as Al₂O₃ or MnO₂, for example the having the composition 31.5% by weight of ZrO₂, 50% by weight of NiO, 17% by weight of CuO and 1.5% by weight of MoO₃, or oxide mixtures which are disclosed in EP-A-963 975 and before reduction with hydrogen comprise from 22 to 40% by weight of ZrO₂, from 1 to 30% by weight of oxygen-comprising compounds of copper, calculated as CuO, from 15 to 50% by weight of oxygen-comprising compounds of nickel, calculated as NiO, with the molar ratio of Ni:Cu being greater than 1, from 15 to 50% by weight of oxygen-comprising compounds of cobalt, calculated as CoO, from 0 to 10% by weight of oxygen-comprising compounds of aluminum and/or manganese, calculated as Al₂O₃ or MnO₂, and no oxygen-comprising compounds of molybdenum, for example the catalyst having the composition 33% by weight of Zr, calculated as ZrO₂, 28% by weight of Ni, calculated as NiO, 11% by weight of Cu, calculated as CuO, and 28% by weight of Co, calculated as CoO.

The process can be carried out in a continuous or discontinuous mode, e.g. in an autoclave, tube reactor or fixed-bed reactor. The reactor design is also not narrowly critical. The feed thereto may be upflowing or downflowing, and design features in the reactor which optimize plug flow in the reactor may be employed.

In context with the present invention, the degree of amination is equal to or greater than 55%, preferably in the range of from 60 to 95%, more preferably of from 65 to 90%, and even more preferably of from 70 to 85%.

Unless specified otherwise herein, the degree of amination is calculated from the total amine value (AZ) divided by sum of the total acetylables value (AC) and tertiary amine value (tert. AZ) multiplicated by 100:

(Total AZ:(AC+tert.AZ)×100).

The total amine value (AZ) is determined according to DIN 16945.

The total acetylables value (AC) is determined according to DIN 53240.

The secondary and tertiary amine are determined according to ASTM D2074-07.

the Primary Amines Value is Calculated as Follows: Primary Amine Value=AZ−Secondary+Tertiary Amine Value.

Primary amine in % of total amine is calculated as follows:

Primary amine in %=((AZ−secondary+tertiary amine value)/AZ)*100

Degree of amination is calculated from(total acetylables value−hydroxyl value)/total acetylables value.

The hydroxyl value is calculated from(total acetylables value+tertiary amine value)−total amine value.

In another preferred embodiment, the etheramines of the invention can also be further reacted with an acid. The acid may be selected from the group consisting of citric acid, lactic acid, sulfuric acid, methanesulfonic acid, hydrogen chloride, phosphoric acid, formic acid, acetic acid, propionic acid, valeric acid, oxalic acid, succinic acid, adipic acid, sebacic acid, glutaric acid, glucaric acid, tartaric acid, malic acid, benzoic acid, salicylic acid, phthalic acid, oleic acid, stearic acid, caproic acid, caprylic acid, capric acid, lauric acid, myristic acid, palmitic acid, linoleic acid and mixtures thereof. In another embodiment the acid may be selected from the group consisting of caproic acid, caprylic acid, capric acid, lauric acid, and myristic acid. In an alternative embodiment, the etheramines of the invention may, in protonated form, have a surfactant as a counter ion, as obtained from e.g. linear alkyl benzene sulphonic acid.

### Applications:

The inventive etheramine mixtures obtained by reductive cyanoethylation may be used in personal care, especially in shampoo and body wash formulations.

They may also be used as curing agent for epoxy resins or as a reactant in the production of polymers but also in polyurethanes, polyureas, epoxy resins, polyamides.

The inventive polyetheramines have proved to be effective for removal of stains, particularly grease, from soiled material. Besides, cleaning compositions with inventive polyetheramines also do not have the cleaning negatives seen with conventional, amine cleaning compositions for hydrophilic bleachable stains, such as coffee, tea, wine, or particulates. Additionally, for stain removal from white fabric, cleaning compositions with inventive polyetheramines do not cause the whiteness negatives that commercially available, amine cleaning compositions cause.

A further advantage of cleaning compositions comprising the inventive polyetheramines is their ability to remove grease stains in cold water cleaning solutions, via pretreatment of the grease stain outside the washing machine, followed by cold water washing. Without being limited by theory, cold water solutions have the effect of causing greases to harden or solidify, making greases more resistant to removal, especially from fabric. Cleaning compositions with with etheramine mixtures according to Formula (I) and/or (II) however, are surprisingly effective when used in pretreatment followed by cold water cleaning.

As used herein the phrase “cleaning composition” includes compositions and formulations designed for cleaning soiled material. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, postrinse fabric treatment, ironing aid, unit dose formulation, delayed delivery formulation, liquid hand dishwashing composition, detergent contained on or in a porous substrate or nonwoven sheet, automatic dish-washing agent, hard surface cleaner, and other suitable forms that may be apparent to one skilled in the art in view of the teachings herein.. Such compositions may be used as a pre-laundering treatment, a post-laundering treatment, may be added during the rinse or wash cycle of the laundering operation, or used in homecare cleaning applications. The cleaning compositions may have a form selected from liquid, powder, single-phase or multiphase unit dose, pouch, tablet, gel, paste, bar, or flake.

The cleaning compositions described herein may include from about 0.1% to about 10%, in some examples, from about 0.2% to about 5%, and in other examples, from about 0.5% to about 3%, by weight the composition, of an amine-terminated polyalkylene glycol of Formula I and/or II.

The inventive etheramine mixtures are effective for removal of stains, particularly grease, from soiled material. Cleaning compositions containing the amine-terminated polyalkylene glycols of the invention also do not exhibit the cleaning negatives seen with conventional amine-containing cleaning compositions on hydrophilic bleachable stains, such as coffee, tea, wine, or particulates. Additionally, unlike conventional amine-containing cleaning compositions, the amine-terminated polyalkylene glycols of the invention do not contribute to whiteness negatives on white fabrics.

A further advantage of cleaning compositions containing the inventive etheramine mixture is their ability to remove grease stains in cold water, for example, via pretreatment of a grease stain followed by cold water washing. Without being limited by theory, it is believed that cold water washing solutions have the effect of hardening or solidifying grease, making the grease more resistant to removal, especially on fabric. Cleaning compositions containing the amine-terminated polyalkylene glycols of the invention are surprisingly effective when used as part of a pretreatment regimen followed by cold water washing.

### Surfactant System

The cleaning compositions comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. The surfactant system may comprise a detersive surfactant selected from anionic surfactants, nonionic surfactants, cationic surfactants, zwitterionic surfactants, amphoteric surfactants, ampholytic surfactants, and mixtures thereof. Those of ordinary skill in the art will understand that a detersive surfactant encompasses any surfactant or mixture of surfactants that provide cleaning, stain removing, or laundering benefit to soiled material.

### Adjunct Cleaning Additives

The cleaning compositions of the invention may also contain adjunct cleaning additives. Suitable adjunct cleaning additives include builders, structurants or thickeners, clay soil removal/anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, hueing agents, dye transfer inhibiting agents, chelating agents, suds supressors, softeners, and perfumes.

### Methods of Use

The present invention includes methods for cleaning soiled material. As will be appreciated by one skilled in the art, the cleaning compositions of the present invention are suited for use in laundry pretreatment applications, laundry cleaning applications, and home care applications. Such methods include, but are not limited to, the steps of contacting cleaning compositions in neat form or diluted in wash liquor, with at least a portion of a soiled material and then optionally rinsing the soiled material. The soiled material may be subjected to a washing step prior to the optional rinsing step.

For use in laundry pretreatment applications, the method may include contacting the cleaning compositions described herein with soiled fabric. Following pretreatment, the soiled fabric may be laundered in a washing machine or otherwise rinsed.

Machine laundry methods may comprise treating soiled laundry with an aqueous wash solution in a washing machine having dissolved or dispensed therein an effective amount of a machine laundry cleaning composition in accord with the invention. An “effective amount” of the cleaning composition means from about 20 g to about 300 g of product dissolved or dispersed in a wash solution of volume from about 5 L to about 65 L. The water temperatures may range from about 5° C. to about 100° C. The water to soiled material (e.g., fabric) ratio may be from about 1:1 to about 20:1. In the context of a fabric laundry composition, usage levels may also vary depending not only on the type and severity of the soils and stains, but also on the wash water temperature, the volume of wash water, and the type of washing machine (e.g., top-loading, front-loading, top-loading, vertical-axis Japanese-type automatic washing machine).

The cleaning compositions herein may be used for laundering of fabrics at reduced wash temperatures. These methods of laundering fabric comprise the steps of delivering a laundry cleaning composition to water to form a wash liquor and adding a laundering fabric to said wash liquor, wherein the wash liquor has a temperature of above 0° C. to about 20° C., or to about 15° C., or to about 10° C. The fabric may be contacted to the water prior to, or after, or simultaneous with, contacting the laundry cleaning composition with water.

Another method includes contacting a nonwoven substrate impregnated with an embodiment of the cleaning composition with soiled material. As used herein, “nonwoven substrate” can comprise any conventionally fashioned nonwoven sheet or web having suitable basis weight, caliper (thickness), absorbency, and strength characteristics. Non-limiting examples of suitable commercially available nonwoven substrates include those marketed under the tradenames SONTARA® by DuPont and POLYWEB® by James River Corp.

Hand washing methods, and combined handwashing with semiautomatic washing machines, are also included.

### Machine Dishwashing Methods

Methods for machine-dishwashing or hand dishwashing soiled dishes, tableware, silverware, or other kitchenware, are included. One method for machine dishwashing comprises treating soiled dishes, tableware, silverware, or other kitchenware with an aqueous liquid having dissolved or dispensed therein an effective amount of a machine dishwashing composition in accord with the invention. By an effective amount of the machine dishwashing composition it is meant from about 8 g to about 60 g of product dissolved or dispersed in a wash solution of volume from about 3 L to about 10 L.

One method for hand dishwashing comprises dissolution of the cleaning composition into a receptacle containing water, followed by contacting soiled dishes, tableware, silverware, or other kitchenware with the dishwashing liquor, then hand scrubbing, wiping, or rinsing the soiled dishes, tableware, silverware, or other kitchenware. Another method for hand dishwashing comprises direct application of the cleaning composition onto soiled dishes, tableware, silverware, or other kitchenware, then hand scrubbing, wiping, or rinsing the soiled dishes, tableware, silverware, or other kitchenware. In some examples, an effective amount of cleaning composition for hand dishwashing is from about 0.5 ml. to about 20 ml diluted in water.

### Packaging for the Compositions

The cleaning compositions described herein can be packaged in any suitable container including those constructed from paper, cardboard, plastic materials, and any suitable laminates. An optional packaging type is described in European Application No. 94921505.7.

### Multi-Compartment Pouch Additive

The cleaning compositions described herein may also be packaged as a multi-compartment cleaning composition.

The present invention is further demonstrated and exemplified in the following examples, however, without being limited to the embodiments described in the examples.

### EXAMPLES

¹H-NMR and ¹³C-NMR measurements were carried out in CDCl₃ with a Bruker 400 MHz spectrometer.

Unless specified otherwise herein, the degree of amination is calculated from the total amine value (AZ) divided by sum of the total acetylables value (AC) and tertiary amine value (tert. AZ) multiplicated by 100:

(Total AZ:(AC+tert.AZ)×100).

The total amine value (AZ) is determined according to DIN 16945.

The total acetylables value (AC) is determined according to DIN 53240.

The secondary and tertiary amine are determined according to ASTM D2074-07.

The primary amines value is calculated as follows: primary amine value=AZ−secondary+tertiary amine value.

Primary amine in % of total amine is calculated as follows:

Primary amine in %=((AZ−secondary+tertiary amine value)/AZ)*100

The hydroxyl value is calculated from(total acetylables value+tertiary amine value)−total amine value.

### 1. EXPERIMENTAL PART

### Example 1 a: 1 mol 2-butyl-2-ethyl-1,3-propandiol+2 mol propylene oxide

In a 2 l autoclave 495.7 g 2-butyl-2-ethyl-1,3-propane diol and 1.7 g potassium tert.-butylate were mixed. The autoclave was purged three times with nitrogen and heated to 140° C. 359.3 g propylene oxide was added within 5 hours. The mixture was allowed to post-react for 4 hours at 140° C. The reaction mixture was stripped with nitrogen and volatile compounds were removed in vacuo at 80° C. The catalyst was removed by adding 26.2 g Macrosorb MP5plus, stirring at 100° C. for 2 hours and filtration. A yellowish oil was obtained (873.0 g, hydroxyl value: 386.6 mgKOH/g).

### Example 1 b: 1 mol 2-butyl-2-ethyl-1,3-propandiol+2 mol propylene oxide+2 mol acrylonitrile

In a 4-neck vessel with stirrer, thermometer, reflux condenser, nitrogen inlet, and dropping funnel 276.4 g of product from example 1 a was placed. 2.3 g of a 50% aqueous solution of tetrakis (2-hydroxyethyl) ammonium hydroxide was added at room temperature. The mixture was heated to 60° C. and 109.3 g acrylonitrile was added dropwise at 60° C. After stirring for 1.5 hours at given temperature the mixture was stirred for additional 14 hours at room temperature. The reaction product was filtered and excess acrylonitrile was removed in vacuo. An orange liquid was obtained (370.0 g, water 0.1%). Complete conversion of acrylonitrile was detected by ¹H-NMR in CDCl₃.

### Example 1 c: 1 mol 2-butyl-2-ethyl-1,3-propandiol+2 mol propylene oxide+2 mol acrylonitrile, Hydrogenated

Representative Procedure for the hydrogenation of cyanoethylated polyoxyalkylenes:

The hydrogenation of example 1 b was conducted in a tubular reactor (length 500 mm, diameter 18 mm) filled with a splitted cobalt catalyst prepared as described in EP636409.

At a temperature of 110° C. and a pressure of 160 bar the nitrile (20 wt.-% in THF) was together with ammonia and hydrogen continuously fed into the reactor at such a rate that full conversion of the nitrile was assured. The crude material was collected and stripped on a rotary evaporator to remove excess ammonia, light weight amines and THF to afford the hydrogenated material. The analytical data of the reaction product are shown below.

| | Secondary + tertiary | |
|---|---|---|
| Total amine value | amine value | Tertiary amine value |
| [mg KOH/g] | [mg KOH/g] | [mg KOH/g] |
| 264.8 | 1.17 | 0.66 |

### Example 2 a: 1 mol 2-butyl-2-ethyl-1,3-propandiol+4 mol propylene oxide

In a 2 l autoclave 411.0 g 2-butyl-2-ethyl-1,3-propane diol and 2.0 g potassium tert.-butylate were mixed. The autoclave was purged three times with nitrogen and heated to 140° C. 596.8 g propylene oxide was added within 8 hours. The mixture was allowed to post-react for 6 hours at 140° C. The reaction mixture was stripped with nitrogen and volatile compounds were removed in vacuo at 80° C. The catalyst was removed by adding 30.2 g Macrosorb MP5plus, stirring at 100° C. for 2 hours and filtration. A yellowish oil was obtained (1001.0 g, hydroxyl value: 273.1 mgKOH/g).

### Example 2 b: 1 mol 2-butyl-2-ethyl-1,3-propandiol+4 mol propylene oxide+1.2 mol acrylonitrile

In a 4-neck vessel with stirrer, thermometer, reflux condenser, nitrogen inlet, and dropping funnel 314.1 g of product from example 2 a was placed. 6.0 g of a 50% aqueous solution of tetrakis (2-hydroxyethyl) ammonium hydroxide was added at room temperature. The mixture was heated to 60° C. and 89.0 g acrylonitrile was added dropwise at 60° C. After stirring for 3 hours at given temperature the mixture was stirred for additional 14 hours at room temperature. The reaction product was filtered and excess acrylonitrile was removed in vacuo. An orange liquid was obtained (354.0 g). Complete conversion of acrylonitrile was detected by ¹H-NMR in CDCl₃. The degree of functionalization with acrylonitrile was detected by ¹H-NMR in CDCl₃ (peak at 2.6 ppm).

### Example 2 c: 1 mol 2-butyl-2-ethyl-1,3-propandiol+4 mol propylene oxide+1.2 mol acrylonitrile, Hydrogenated

Example 2 b was hydrogenated according to the representative procedure described in example 1 c. The analytical data of the reaction product are shown below.

| Total | | |
|---|---|---|
| amine value | Secondary + tertiary amine value | Tertiary amine value |
| [mg KOH/g] | [mg KOH/g] | [mg KOH/g] |
| 145.2 | 3.83 | 3.19 |

### Comparative Example 3 a: 1 mol 2-butyl-2-ethyl-1,3-propandiol+4 mol propylene oxide

In a 2 l autoclave 322.6 g 2-Butyl-2-ethyl-1,3-propane diol and 7.9 g KOH (50% in water) were mixed and stirred under vacuum (<10 mbar) at 120° C. for 2 h. The autoclave was purged with nitrogen and heated to 140° C. 467.8 g propylene oxide was added in portions within 6 h. To complete the reaction, the mixture was allowed to post-react for additional 5 h at 140° C. The reaction mixture was stripped with nitrogen and volatile compounds were removed in vacuo at 80° C. The catalyst potassium hydroxide was removed by adding 2.3 g synthetic magnesium silicate (Macrosorb MP5plus, Ineos Silicas Ltd.), stirring at 100° C. for 2 h and filtration. A yellowish oil was obtained (772.0 g, hydroxyl value: 248.5 mgKOH/g).

### Comparative Example 3 b: 1 mol 2-butyl-2-ethyl-1,3-propandiol+4 mol propylene oxide, Aminated

In a 9 l autoclave 600 g of the resulting diol mixture from example 3 a, 1250 g THF and 1500 g ammonia were mixed in presence of 200 ml of a solid catalyst as described in EP0696572B1. The catalyst containing nickel, cobalt, copper, molybdenum and zirconium was in the form of 3×3 mm tables. The autoclave was purged with hydrogen and the reaction was started by heating the autoclave. The reaction mixture was stirred for 18 h at 205° C., the total pressure was maintained at 270 bar by purging hydrogen during the entire reductive amination step. After cooling down the autoclave the final product was collected, filtered, vented of excess ammonia and stripped in a rotary evaporator to remove light amines and water. A total of 560 grams of a low-color etheramine mixture was recovered. The analytical results thereof are shown below.

| Total | Total | Secondary | Tertiary | | | Primary |
|---|---|---|---|---|---|---|
| amine- | acetylatables | and tertiary | amine- | Hydroxyl | Degree of | Amine value |
| value | value | amine value | value | value | amination | in % of total |
| mg KOH/g | mg KOH/g | mg KOH/g | mg KOH/g | mg KOH/g | in % | amine |
| 278.21 | 287.70 | 6.96 | 4.60 | 14.09 | 95.18 | 97.50 |

### Comparative Example 4 a: 1 mol 2-butyl-2-ethyl-1,3-propandiol+5.6 mol propylene oxide

In a 2 l autoclave 313.1 g 2-Butyl-2-ethyl-1,3-propane diol and 3.8 g KOH (50% in water) were mixed and stirred under vacuum (<10 mbar) at 120° C. for 2 h. The autoclave was purged with nitrogen and heated to 140° C. 635.6 g propylene oxide was added in portions within 6 h. To complete the reaction, the mixture was allowed to post-react for additional 5 h at 140° C. The reaction mixture was stripped with nitrogen and volatile compounds were removed in vacuo at 80° C. The catalyst was removed by adding 50.9 g water and 8.2 g phosphoric acid (40% in water) stirring at 100° C. for 0.5 h and dewatering in vacuo for 2 hours. After filtration 930.0 g of a light yellowish oil was obtained (hydroxyl value: 233 mgKOH/g).

### Comparative Example 4 b: 1 mol 2-butyl-2-ethyl-1,3-propandiol+5.6 mol propylene oxide, partially Aminated

The amination of example 4 a was conducted in a tubular reactor (length 500 mm, diameter 18 mm) which had been charged with 15 mL of silica (3×3 mm pellets) followed by 70 mL (74 g) of the catalyst precursor (containing oxides of nickel, cobalt, copper and tin on gama-Al₂O₃, 1.0-1.6 mm split—prepared according to WO 2013/072289 A1) and filled up with silica (ca. 15 mL).

The catalyst was activated at atmospheric pressure by being heated to 100° C. with 25 norm litre (NI)/h of nitrogen, then 3 hours at 150° C. in which the hydrogen feed was increased from 2 to 25 NI/h, then heated to 280° C. at a heating rate of 60° C. per hour and kept at 280° C. for 12 hours. The reactor was cooled to 100° C., the nitrogen flow was turned off and the pressure was increased to 120 bar.

The catalyst was flushed with ammonia at 100° C., before the temperature was increased to 184° C. and the alcohol feed was started with a WHSV of 0.44 kg/liter*h (molar ratio ammonia/alcohol=27:1, hydrogen/alcohol=6:1). The crude material was collected and stripped on a rotary evaporator to remove excess ammonia, light weight amines and reaction water to afford the aminated material. The analytical data of the reaction product is shown below.

| Total | Total | Secondary | Tertiary | | | Primary |
|---|---|---|---|---|---|---|
| amine- | acetylatables | and tertiary | amine- | Hydroxyl | Degree of | Amine value |
| value | value | amine value | value | value | amination | in % of total |
| mg KOH/g | mg KOH/g | mg KOH/g | mg KOH/g | mg KOH/g | in % | amine |
| 111.80 | 236.80 | 0.14 | 0.00 | 125.00 | 47.21 | 99.87 |

### Comparative Example 5 b: 1 mol 2-butyl-2-ethyl-1,3-propandiol+4 mol propylene oxide+0.8 mol acrylonitrile

In a 4-neck vessel with stirrer, thermometer, reflux condenser, nitrogen inlet, and dropping funnel 392.1 g of product from example 2 a was placed. 4.7 g of a 50% aqueous solution of tetrakis (2-hydroxyethyl) ammonium hydroxide was added at room temperature. The mixture was heated to 60° C. and 61.3 g acrylonitrile was added dropwise at 60° C. After stirring for 6 hours at given temperature the mixture was stirred for additional 14 hours at room temperature. The reaction product was filtered and excess acrylonitrile was removed in vacuo. An orange liquid was obtained (414.0 g). Complete conversion of acrylonitrile was detected by ¹H-NMR in CDCl₃. The degree of functionalization with acrylonitrile was detected by ¹H-NMR in CDCl₃ (peak at 2.6 ppm).

### Comparative Example 5 c: 1 mol 2-butyl-2-ethyl-1,3-propandiol+4 mol propylene oxide+0.8 mol acrylonitrile, Hydrogenated

Compound 5 b was hydrogenated over Raney cobalt in a continuously operated autoclave. At a temperature of 110° C. and a pressure of 160 bar the nitrile (10 wt.-% in ethanol) was together with ammonia and hydrogen continuously fed into the reactor at such a rate that full conversion of the nitrile was assured (controlled by NMR spectroscopy). The crude material was collected and stripped on a rotary evaporator to remove excess ammonia, light weight amines and ethanol to afford the hydrogenated material. The analytical data of the reaction product is shown below.

| Total | Total | Secondary | Tertiary | | | Primary |
|---|---|---|---|---|---|---|
| amine- | acetylatables | and tertiary | amine- | Hydroxyl | Degree of | Amine |
| value | value | amine value | value | value | amination | in % of total |
| mg KOH/g | mg KOH/g | mg KOH/g | mg KOH/g | mg KOH/g | in % | amine |
| 90.24 | 278.8 | 1.98 | 1.66 | 190.2 | 32.2 | 97.8 |

### Example 6 b: 1 mol 2-butyl-2-ethyl-1,3-propandiol+4 mol propylene oxide+2 mol acrylonitrile

In a 4-neck vessel with stirrer, thermometer, reflux condenser, nitrogen inlet, and dropping funnel 39.3 g of product from example 2 a was placed. 0.23 g of a 50% aqueous solution of tetrakis (2-hydroxyethyl) ammonium hydroxide was added at room temperature. The mixture was heated to 60° C. and 10.9 g acrylonitrile was added dropwise at 60° C. After stirring for 1.5 hours at given temperature the mixture was stirred for additional 14 hours at room temperature. The reaction product was filtered and excess acrylonitrile was removed in vacuo. An orange liquid was obtained (36.5 g). Complete conversion of acrylonitrile was detected by ¹H-NMR in CDCl₃.

### Example 6 c: 1 mol 2-butyl-2-ethyl-1,3-propandiol+4 mol propylene oxide+2 mol acrylonitrile, Hydrogenated

Example 6 b was hydrogenated according to the representative procedure described in example 1 c. The analytical data of the reaction product are shown below.

| Total | | |
|---|---|---|
| amine value | Secondary + tertiary amine value | Tertiary amine value |
| [mg KOH/g] | [mg KOH/g] | [mg KOH/g] |
| 204.70 | 1.21 | 1.09 |

### Example 7 a: 1 mol 2-ethyl-1,3-hexane diol+4 mol propylene oxide

In a 2 l autoclave 150.0 g 2-ethyl-1,3-hexane diol and 1.5 g potassium hydroxide (50% aqueous solution) were mixed. The mixture was heated to 110° C. and the water was removed in vacuo for 2 hours at 30 mbar. The autoclave was purged three times with nitrogen and heated to 140° C. 231.2 g propylene oxide was added within 2 hours. The mixture was allowed to post-react for 4 hours at 140° C. The reaction mixture was stripped with nitrogen and volatile compounds were removed in vacuo at 80° C. The catalyst was removed by adding 11.4 g Macrosorb MP5plus, stirring at 100° C. for 2 hours and filtration. A yellowish oil was obtained (373.6 g, hydroxyl value: 245.0 mgKOH/g).

### Example 7 b: 1 mol 2-ethyl-1,3-hexane diol+4 mol propylene oxide+2 mol acrylonitrile

In a 4-neck vessel with stirrer, thermometer, reflux condenser, nitrogen inlet, and dropping funnel 151.3 g of product from example 7 was placed. 3.6 g of a 50% aqueous solution of tetrakis (2-hydroxyethyl) ammonium hydroxide was added at room temperature. The mixture was heated to 60° C. and 64.1 g acrylonitrile was added dropwise at 60° C. After stirring for 3 hours at given temperature the mixture was stirred for additional 14 hours at room temperature. The reaction product was filtered and excess acrylonitrile was removed in vacuo. An orange liquid was obtained (174.0 g). Complete conversion of acrylonitrile was detected by ¹H-NMR in CDCl₃. The degree of functionalization with acrylonitrile was detected by ¹H-NMR in CDCl₃ (peak at 2.6 ppm).

### Example 7 c: 1 mol 2-ethyl-1,3-hexane diol+4 mol propylene oxide+2 mol acrylonitrile, Hydrogenated

Example 7 b was hydrogenated according to the representative procedure described in example 1 c. The analytical data of the reaction product are shown below.

| Total | | |
|---|---|---|
| amine value | Secondary + tertiary amine value | Tertiary amine value |
| [mg KOH/g] | [mg KOH/g] | [mg KOH/g] |
| 218.7 | 5.76 | 1.02 |

### Example 8 b: 1 mol 2-ethyl-1,3-hexane diol+4 mol propylene oxide+1.2 mol acrylonitrile

In a 4-neck vessel with stirrer, thermometer, reflux condenser, nitrogen inlet, and dropping funnel 151.3 g of product from example 7 a was placed. 4.8 g of a 50% aqueous solution of tetrakis (2-hydroxyethyl) ammonium hydroxide was added at room temperature. The mixture was heated to 60° C. and 29.3 g acrylonitrile was added dropwise at 60° C. After stirring for 3 hours at given temperature the mixture was stirred for additional 14 hours at room temperature. The reaction product was filtered and excess acrylonitrile was removed in vacuo. An orange liquid was obtained (160.0 g). Complete conversion of acrylonitrile was detected by ¹H-NMR in CDCl₃. The degree of functionalization with acrylonitrile was detected by ¹H-NMR in CDCl₃ (peak at 2.6 ppm).

### Example 8 c: 1 mol 2-ethyl-1,3-hexane diol+4 mol propylene oxide+1.2 mol acrylonitrile, Hydrogenated

Example 8 b was hydrogenated according to the representative procedure described in example 1 c. The analytical data of the reaction product are shown below.

| Total | | |
|---|---|---|
| amine value | Secondary + tertiary amine value | Tertiary amine value |
| [mg KOH/g] | [mg KOH/g] | [mg KOH/g] |
| 146.9 | 4.52 | 0.82 |

### 2. Use as Additives in Laundry Detergent

### 2.1 Stain Removal Index

Technical stain swatches of blue knitted cotton containing grease stains were purchased from Warwick Equest Ltd. The stains were washed for 30 min in a launder-o-meter (manufactured by SDL Atlas) at room temperature using per canister 500 mL of washing solution, 20 steel balls (weight of 1 ball is 1 g) and ballast fabrics. The washing solution contained 5000 ppm of detergent composition DC1 (table 1). Water hardness was 2.5 mM (Ca²⁺:Mg²⁺ molar ratio was 4:1). Additives were added to the washing solution of each canister separately and in the amount as detailed below. After addition the pH value was re-adjusted to the pH value of washing solution without additive.

Standard colorimetric measurement was used to obtain L*, a* and b* values for each stain before and after the washing. From L*, a* and b* values the stain level were calculated as color difference ΔE (calculated according to DIN EN ISO 11664-4) between stain and untreated fabric.

Stain removal from the swatches was calculated as follows:

$$Stain Removal Index ( SRI ) = \frac{( Δ {E}_{initial} - Δ {E}_{washed} )}{Δ {E}_{initial}} \times 100$$

ΔEᵢₙᵢₜᵢₐₗ=Stain level before washing

ΔE_{washed}=Stain level after washing

Stain level corresponds to the amount of grease on the fabric. The stain level of the fabric before the washing (ΔEᵢₙₜᵢₐₗ) is high, in the washing process stains are removed and the stain level after washing is smaller (ΔE_{washed}). The better the stains have been removed the lower the value for ΔE_{washed} will be and the higher the difference will be to ΔEᵢₙₜᵢₐₗ. Therefore, the value of stain removal index increases with better washing performance.

TABLE 1
Detergent composition DC1

| Ingredients of liquid detergent composition DC1 | percentage by weight |
|---|---|
| n-C10-C13-alkylbenzene sulfonic acid | 5.3 |
| coconut C12-C18 fatty acid | 2.4 |
| sodium laureth sulfate + 2 EO | 7.7 |
| potassium hydroxide | 2.2 |
| C13C15-oxo alcohol + 7 EO | 5.4 |
| 1,2 propylene glycol | 6 |
| ethanol | 2 |
| water | to balance |
| pH of detergent composition DC1 = 8.4 | |

TABLE 2
Washing Test 1:

| | | SRI, |
|---|---|---|
| | additive/ | stain: Beef |
| | [g] | Fat |
| without additive | — | 25.6 |
| with Example 1 c | 0.0375 | 36.9 |
| with Comparitive Example 3 b | 0.0375 | 32.1 |
| with Comparitive Example 5 c | 0.0375 | 29.5 |

TABLE 3
Washing Test 2:

| | | SRI, |
|---|---|---|
| | additive/ | stain: Chicken |
| Additive | [g] | Fat |
| without additive | — | 24.3 |
| with Example 2 c | 0.0375 | 33.1 |
| with Comparitive Example 4 b | 0.0375 | 28.0 |

All washing tests with Examples 1 and 2 (Table 2 and Table 3) show improved stain removal compared to Comparative Examples 3, 4, and 5.

### 2.2 Anti-Redeposition Methodology

The Anti-Redeposition Methodology uses a grease-soluble fluorescent dye to mimic the redeposition behavior of grease. To summarize the method, the dye is incorporated in bacon grease and the fluorescent dye-doped bacon grease is applied to fabrics to create a fluorescent grease stain. The fluorescent grease stain is incorporated into a wash system with a NIL-polymer detergent. The grease stains are washed for 12 mins. This creates a suspension of fluorescent grease. Then the wash system is paused and the anti-redeposition technology is added to the wash system. The wash system is stirred and clean white tracer fabrics are added to the wash system and the wash is restarted. At the end of the wash the tracers are removed from the wash, dried and evaluated to measure the fluorescent signal on the tracer fabrics. The intensity of the fluorescent signal is correlated to the power of the anti-redeposition technology to suspend greasy soils. The lower the intensity of the fluorescent signal on the tracer fabric, the greater the power of the anti-redeposition technology for suspending grease.

To make stains with bacon grease doped with fluorescent dye start by doping the fluorescent dye into bacon grease. The dye doped grease is made at least one day before applying to fabric. To solubilize the fluorescent dye (Fluorol 555 CAS#19125-99-6, from Exciton Dayton, Ohio), into the grease, completely melt bacon grease (from EMC) in a 50 OC oven or water bath. Then add 0.1 g of Fluorol 555 to 100 g melted bacon grease and stir. If possible, it is ideal to follow with sonication will improve the uniformity of solubilization and this will reduce noise level of the method.

After at least one day, heat the bacon grease again in a 50° C. oven or water bath until completely melted. Lay fabric swatches (cotton or polycotton) out onto a weight cup to keep fabric suspended. Suspending the fabric over a cup, stops grease loss from bleeding through the fabric onto another surface while the grease cools on the fabric. With a 1 ml pipette apply three 250 ul spots (approximately 0.25 g) to 1 cotton swatch spaced far enough to ensure the grease spots remain separate and don't wick into each other. Applying three small stains enables emulsification of the grease during the wash process. Keep the bacon grease fluid while applying, reintroduce into the oven or water bath if the bacon grease begins to congeal to re-fluidize it. Let the stains rest over the weigh cup for several hours until these are thoroughly solidified. If stains will be stored before use, the cooled stains can be layered with wax paper between the stains and wrapped in aluminum foil, then stored in the refrigerator until time to use the stains. It is important to protect the stains from light, and keep these cool to prevent photo-oxidation of the fluorescent dye and oxidation/microbial contamination of the bacon grease. Stains can be stored in this manner for 1 month. Expired stains should be discarded.

TABLE 4
Detergent composition DC2.

| Raw Material | % |
|---|---|
| C12-15 alkyl ethoxy (1.8) sulfate | 11.27 |
| Nonionic C24 EO9¹ | 1.04 |
| DTPA² | 0.30 |
| 1,2 Propylene Glycol | 3.72 |
| Monoethanolamine | 3.17 |
| Sodium Hydroxide | 0.69 |
| Sodium Tetraborate | 2.03 |
| Alkyl benzene sulfonic acid | 10.01 |
| Sodium Formate | 0.11 |
| Citric Acid | 2.87 |
| C1218 Fatty Acid | 1.11 |
| Calcium Formate | 0.10 |
| Ethanol | 1.49 |
| Water | Balance to 100% |

¹Nonionic 24-9 is a C12-14 alcohol ethoxylate, with an average degree of ethoxylation of 9
²DTPA is diethylenetetraamine pentaacetic acid

To create the wash suspension of fluorescent dye-doped grease, start with a 5.29 g of a detergent containing surfactant and NIL-polymer (detergent composition DC2 of Table 4). Add two soiled fabrics with 3 spots each of dyed grease (approximately 1.5 g of fluorescent dye-doped grease) in 7.5 L of water in a mini-washer washing machine tub. Add 200 g of clean terry ballast. Set wash conditions at 30.5° C., 119.7 ppm of a 3:1 Ca²⁺/Mg²⁺ (0.98 moles Ca²⁺) mix to simulate water hardness.

After 12 min., remove the dyed grease stains and add the three polycotton 50/50 tracers and the anti-redeposition technology and agitate for 30 s. After agitation, complete another 12 min. wash cycle and 2 min. rinse (21.1° C.). Dry each test load plus ballast (in separate dryers) for 50 min (normal dryer cycle). To improve signal sensitivity, run the test swatches through 2 more cycles w/the test technology to created 3 cycles of deposition of dyed grease.

Cut samples from the traces to fit into a 12-24 well plate. Place cut samples in the bottom of the well and use o-rings to hold the swatches flat in the well. Read 3-6 spots per treatment. Sample the most uniform, representative parts of the tracer swatch. Read the fluorescence intensity on the samples using a reflectance fluorescence spectrometer (BMG Fluostar).

Index the test samples to the control samples (NIL-polymer detergent) equation: (test signal−control signal)/control signal=index. If the index is a negative number, the test sample provides improved anti-redeposition vs. the control. If the test sample is positive, it is causing deposition versus control.

TABLE.5
The index is more negative for examples 1c & 6c over Comparative example 9.
Examples 1c and 6c provide improved anti-redeposition vs. Comparative example 9.

| Molecule | | Anti-redepo- |
|---|---|---|
| Name | Molecular Structure | sition Index |
| Comparative example 9 | 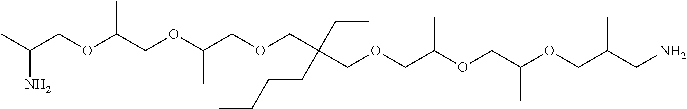 [SMILES: CCCCC(CC)(COCC(C)OCC(C)OCC(C)N)COCC(C)OCC(C)OCC(C)CN] | −14.75 |
| Example 1c; (2-Butyl-2- Ethyl-Propane- diol)(PO/OH)1 (CAN/)H)1 hydrogenated | 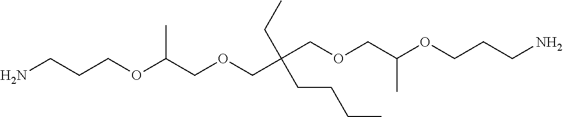 [SMILES: CCCCC(CC)(COCC(C)OCCCN)COCC(C)OCCCN] | −17.14 |
| Example 6c (2-Butyl-2- Ethyl-Propane- diol)(PO/OH)2 (CAN/)H)1 hydrogenated | 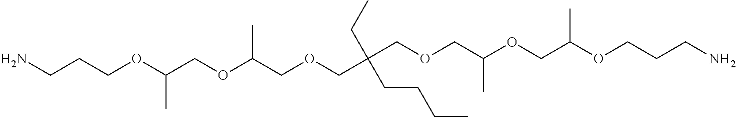 [SMILES: CCCCC(CC)(COCC(C)OCC(C)OCCCN)COCC(C)OCC(C)OCCCN] | −19.88 |

Comparative example 9 was prepared by a reaction sequence similar to the one described for comparative example 4b but conditions were adapted to obtain a high amination degree.

1. An etheramine mixture comprising at least 90% by weight, based on the total weight of the etheramine mixture, of an amine of Formula (I) and/or (II), 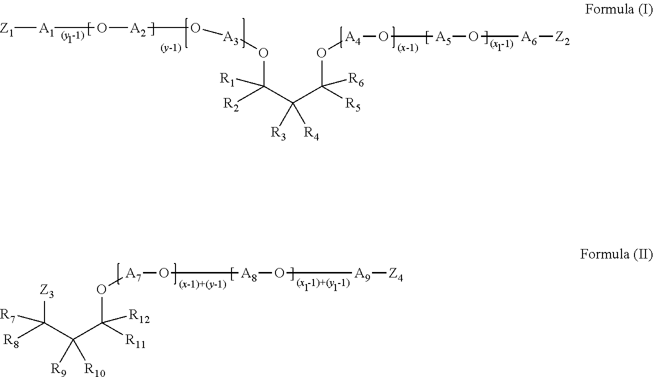
wherein R₁-R₁₂ are independently selected from H, alkyl, cycloalkyl, aryl, alkylaryl, or arylalkyl, wherein at least one of R₁-R₆ and at least one of R₇-R₁₂ is different from H,
wherein A₁-A₉ are independently selected from linear or branched alkanediyl groups having 2 to 18 carbon atoms,
wherein Z₁-Z₄ are independently selected from —OH and linear —OCH₂CH₂CH₂NH₂, wherein the degree of amination of each of the etheramines of Formula (I) and Formula (II) is equal to or greater than 50%, and wherein the sum of x+y is in the range of from 2 to 200, wherein x≥1 and y≥1; and x₁+y₁ is in the range of from 2 to 200, wherein x₁≥1 and y₁≥1.

2. The etheramine mixture according to claim 1, wherein the etheramine mixture comprises at least 95% by weight, based on the total weight of the etheramine mixture, of the amine of Formula (I) and/or (II).

3. The etheramine mixture according to claim 1, wherein in said polyetheramine of Formula (I) or Formula (II), x+y is in the range of from 2 to 20.

4. The etheramine mixture according to claim 1, wherein in said polyetheramine of Formula (I) or Formula (II), x+y is in the range of from 3 to 20.

5. The etheramine mixture according to claim 1, wherein in said polyetheramine of Formula (I) or Formula (II), the degree of amination lies in the range of from 60% to 95%.

6. The etheramine mixture according to claim 1, wherein in said polyetheramine of Formula (I) or Formula (II), A₁-A₉ are independently selected from the group consisting of ethylene, propylene, or butylene.

7. The etheramine mixture according to claim 1, wherein in said polyetheramine of Formula (I) or Formula (II), each of A₁-A₉ is propylene.

8. The etheramine mixture according to claim 1, wherein in said polyetheramine of Formula (I) or Formula (II), R₁, R₂, R₅, R₆, R₇, R₈, R₁₁, and R₁₂ are H and R₃, R₄, R₉, and R₁₀ are independently selected from C1-16 alkyl or aryl.

9. The etheramine mixture according to claim 1, wherein in said polyetheramine of Formula (I) or Formula (II), R₁, R₂, R₅, R₆, R₇, R₈, R₁₁, and R₁₂ are H and R₃, R₄, R₉, and R₁₀ are independently selected from a butyl group, an ethyl group, a methyl group, a propyl group, or a phenyl group.

10. The etheramine mixture according to any claim 1, wherein in said polyetheramine Formula (I) or Formula (II), R₃ and R₉ are each an ethyl group, R₁, R₂, R₅ R₆, R₇, R₈, R₁₁, R₁₂ are each H, R₄ and R₁₀ are each a butyl group.

11. The etheramine mixture according to claim 1, wherein the polyetheramine of Formula (I) or Formula (II) has a weight average molecular weight of about 290 to about 1000 grams/mole.

12. The etheramine mixture according to claim 1, wherein the polyetheramine of Formula (I) or Formula (II) is reacted with an acid.

13. A method of using the etheramine mixture of claim 1, the method comprising using the etheramine mixture in personal care.

14. A method of using the etheramine mixture of claim 1, the method comprising using the etheramine mixture in shampoo and body wash formulations.

15. A method of using the etheramine mixture of claim 1, the method comprising using the etheramine mixture as curing agent for epoxy resins or as a reactant in the production of polymers.

16. A method of using the etheramine mixture of claim 1, the method comprising using the etheramine mixture in polyurethanes, polyureas, and as thermoplastic polyamide adhesives.

17. The etheramine mixture according to claim 1, wherein A₁-A₉ are independently selected from linear or branched alkanediyl groups having 2-10 carbon atoms.

18. The etheramine mixture according to claim 17, wherein A₁-A₉ are independently selected from linear or branched alkanediyl groups having 2-5 carbon atoms.

19. The etheramine mixture according to claim 1, wherein and x₁+y₁ is in the range of from 2 to 20.

20. The etheramine mixture according to claim 19, wherein and x₁+y₁ is in the range of from 2 to 10.
